(19) United States
(12) Patent Application Publication
(10) Pub. No.: US 2025/0120657 A1
(43) Pub. Date: Apr. 17, 2025

(54) PATIENT TREATMENT EFFICACY MONITORING

(71) Applicant: Shape Medical Systems, Inc., St. Paul, MN (US)

(72) Inventors: Stephen T. Anderson, North Oaks, MN (US); David M. Anderson, White Bear Lake, MN (US); Dean J. MacCarter, Castle Pines, CO (US)

(21) Appl. No.: 18/898,039

(22) Filed: Sep. 26, 2024

Related U.S. Application Data
(60) Provisional application No. 63/589,397, filed on Oct. 11, 2023.

(51) Int. Cl. A61B 5/00; A61B 5/0205; A61B 5/024; A61B 5/08; A61B 5/145; G16H 20/10; G16H 50/30

(52) U.S. Cl. CPC A61B 5/7267; A61B 5/082; A61B 5/7275; A61B 5/742; G16H 20/10; G16H 50/30; A61B 5/0205; A61B 5/024; A61B 5/14542

(57) ABSTRACT

An example computer-implemented method for diagnosing cause of exertional dyspnea can include: receiving cardiopulmonary exercise test data corresponding to a submaximal cardiopulmonary exercise test performed by patients; gathering, by a computing device, observations to be classified based upon cardiopulmonary exercise test data; extracting, by the computing device, numerical information from the observations; classifying, by the computing device, the numerical information; and generating, by the computing device, an interpretation of the numerical information.

### RELATED APPLICATIONS

This application is related to: U.S. Pat. No. 8,630,811 dated Jan. 14, 2014; U.S. Pat. No. 8,775,093 dated Jul. 8, 2014; U.S. Pat. No. 10,010,264 dated Jul. 3, 2018; and U.S. Pat. No. 11,497,439 dated Nov. 15, 2022; each of which is hereby incorporated by reference in its entirety.

### BACKGROUND

The early symptoms of chronic disease—such as dyspnea, dizziness, and fatigue—are often mild and are common to many other conditions, including deconditioning. At rest, there are often no symptoms and no apparent signs of illness. As a result, diagnosis can be delayed for months or even years meaning that the underlying disease is frequently not recognized until the disease is relatively advanced, thus more difficult to treat.

Pulmonary Hypertension (PH) is hemodynamically classified as pre-capillary (as seen in idiopathic pulmonary hypertension (IPAH)) or post-capillary (as seen in heart failure with either reduced ejection fraction (HFrEF) or heart failure with preserved ejection fraction (HFpEF)). Overlaps in these conditions exist. Some patients present with risk factors for left heart disease but pre-capillary PH, whereas patients with HFpEF may have combined pre- and post-capillary PH. Patients with atypical IPAH share features of both typical IPAH and PH-HFpEF suggesting that there may be a continuum between these conditions.

The non-specific nature of symptoms associated with PH means that the diagnosis cannot be made on symptoms alone. Neither can it be diagnosed using a 6 minute walk test. A series of investigations is required to make an initial diagnosis, to refine that diagnosis in terms of clinical class of a disease, and to evaluate the degree of functional and hemodynamic impairment. Current PH evaluation and classification (type, functional capacity, hemodynamics) methods include blood tests and immunology, HIV test, thoracic/abdominal ultrasound scan, 6-minute walk test (6-MWT), peak VO₂, right heart catheterization, and vaso-reactivity testing. It is with exercise that the sympathetic and neuro-hormonal systems trigger increased vasoconstriction of the pulmonary arteriolar vascular beds, thus causing an elevation in pulmonary vascular resistance and reduced blood flow through the pulmonary vascular circuit and reduced gas exchange at the capillary/alveolar junction. The reduced blood flow is mismatched to the air flow in the bronchioles and alveoli—also known as Ventilation/Perfusion (V/Q) mismatching.

It is often that the exercise state is not evaluated by any pulmonary function parameters that truly represent both lung ventilation and also perfusion (cardiac output) as related to actual gas exchange in the lungs. Adequate gas exchange during exercise is dependent upon and blood flow and breathing reserve. Instead, walking distance and maybe peak oxygen uptake are measured, which are “secondary outcomes” of gas exchange.

The major shortcoming of the existing classification systems for Heart Failure and Pulmonary Hypertension (NYHA and WHO) systems is that they rely on subjective observations by the patient and interpretation of those observations by the physician.

The 6-minute walk test, while simple and convenient, has many limitations including issues relating to reproducibility, sensitivity, and essentially a work plateau in functional assessment when patients have less functional impairment.

The logistics of performing an exercise test to maximal exertion, including laboratory staffing, direct physician supervision and test duration, in addition to the increased level of patient discomfort, does not lend to conducting this procedure in a serial fashion over short time intervals (i.e., several weeks-months). In addition, it has been found that maximum exercise levels are not representative of lower level, activities of daily living.

### SUMMARY

In one aspect, an example computer-implemented method for diagnosing cause of exertional dyspnea can include: receiving cardiopulmonary exercise test data corresponding to a submaximal cardiopulmonary exercise test performed by patients; gathering, by a computing device, observations to be classified based upon cardiopulmonary exercise test data; extracting, by the computing device, numerical information from the observations; classifying, by the computing device, the numerical information; and generating, by the computing device, an interpretation of the numerical information.

### DESCRIPTION OF THE DRAWINGS

FIG. 1 is a schematic drawing that illustrates the functional components of a cardiopulmonary exercise (CPX) testing system usable with the present disclosure.

FIG. 2 is a schematic drawing that illustrates one form of exercise protocol that is used to place a volume load on the cardiopulmonary system.

FIG. 3 illustrates an organization of the measured data once it is acquired from the cardiopulmonary exercise gas exchange analyzer.

FIG. 4 illustrates an example plot showing the PECO₂/PetCO₂ ratio isopleths and the PECO₂ vs PetCO₂ axes and the separation between four disease classes.

FIG. 5 illustrates a graph of a normal PetCO₂ and PECO₂ response to exercise over the course of the exercise test.

FIG. 6 illustrates an unpopulated test plot of the present disclosure.

FIG. 7 illustrates the location of vectors for two CPX tests on the same patient.

FIG. 8 illustrates the location of vectors for more than two CPX tests on the same patient.

FIG. 9 illustrates example logic used to provide the Impressions Statement for explanation of the test results.

FIGS. 10a-d illustrates example populated tests for various physiological conditions or disease states.

FIG. 11 illustrates an example networked CPX testing system.

FIG. 12 illustrates an example plot of GxCap against VO₂ for a patient test.

FIG. 13 illustrates an example plot of GxCap against time for a patient test.

FIG. 14 illustrates an example method for calculating a patient score.

FIG. 15 illustrates an example table with metrics used to calculate the patient score of FIG. 14.

FIG. 16 illustrates another example table with metrics used to calculate the patient score of FIG. 14.

FIG. 17 illustrates an example system programmed to execute the method of FIG. 15.

FIG. 18 is an example user interface of the system of FIG. 17.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

The following detailed description, including the use of patient data, is intended to be exemplary of a preferred method of utilizing the concepts of the present disclosure and is not intended to be exhaustive or limiting in any manner with respect to similar methods and additional or other steps which might occur to those skilled in the art. The following description further utilizes illustrative examples, which are believed sufficient to convey an adequate understanding of the broader concepts to those skilled in the art, and exhaustive examples are believed unnecessary.

### Example Advantages

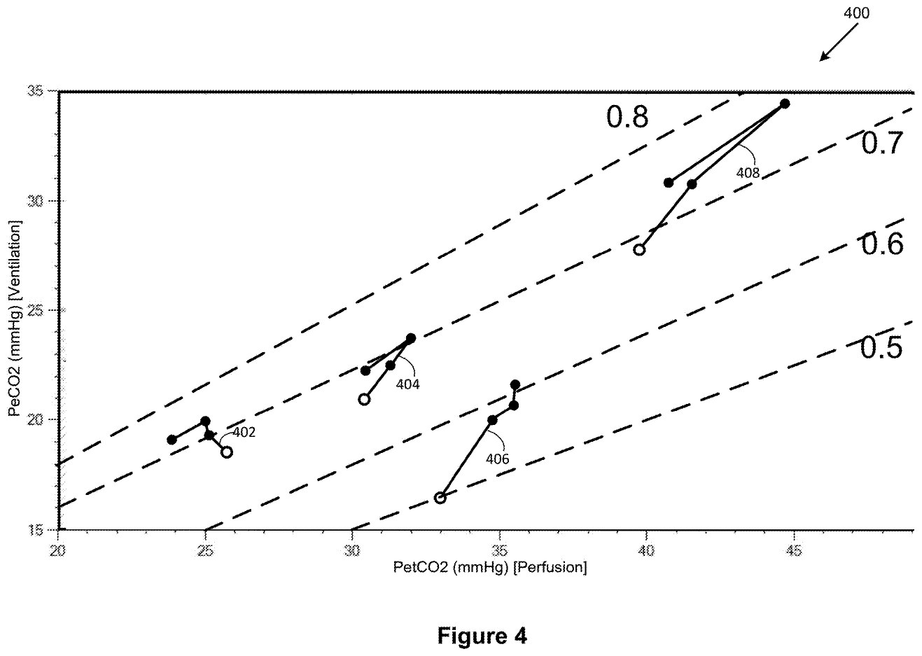

FIG. 4 provides an example plot 400 of test values for PECO₂ (ventilation) and PetCO₂ (perfusion) at rest, during unloaded cycling, at anaerobic threshold, and at end of exercise for various physiologic states or disease types. In the example plot 400, PECO₂ (“y” axis) is plotted against PetCO₂ (“x” axis) for test datapoints with isopleths representing the ratios 0.5, 0.6, 0.7, and 0.8 shown. In this example, a test plot 402 represents example data from a test indicative of a subject with PAH, a test plot 404 represents example data from a test indicative of a subject with LVF, a test plot 406 represents example data from a test indicative of a subject with COPD, and a test plot 408 represents example data from a test indicative of a subject with a normal physiologic state. Each of the test plots 402, 404, 406, and 408 include four connected data points. The first data point (shown as an open circle) corresponds to PECO₂ and PetCO₂ values at rest. The remaining data points correspond (in order) to PECO₂ and PetCO₂ values during unloaded cycling, at anaerobic threshold, and at end of exercise.

The use of plots similar to plot 400 to identify disease types for patients is discussed further in Hansen, James E., et al. “Mixed-expired and end-tidal CO2 distinguish between ventilation and perfusion defects during exercise testing in patients with lung and heart diseases.” Chest 132.3 (2007): 977-983. However, no method for a computerized analysis of the resulting plot is presented or suggested. Also, the use of 4 data points per test may be confusing and unacceptable for clinical use. Beneficially, at least some implementations described herein only require 2 data points—the PECO₂ and PetCO₂ at rest and at peak or at the end of exercise. The resulting vector is further clarified as to length, direction, and mean value of the coordinate points.

Furthermore, how such data can be used to track therapy is not addressed.

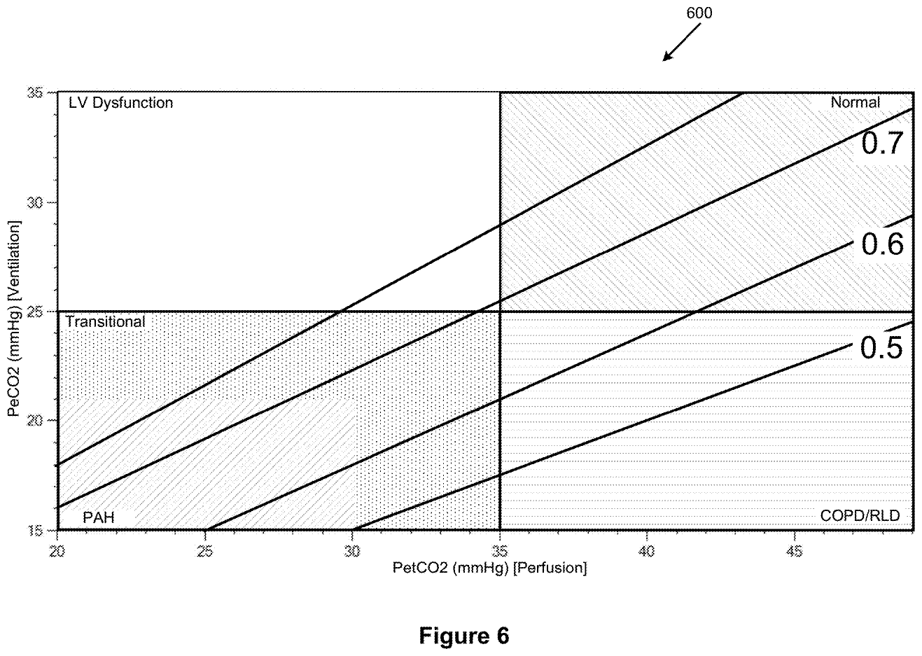

Using the method described below in accordance with the disclosure, the plot as in FIG. 6 uses a format similar to that shown in plot 400 (of FIG. 4) but with clearly defined “zones” that are used to identify the primary suspected disease causing the patient's shortness of breath. Using the measured values collected during a CPX test a single directional vector annotated with the median of the vector point identifies the zone, or disease classification. Thus, the physician's test interpretation is vastly simplified.

General Considerations—The present disclosure includes a pattern recognition system consisting of a) a cardiopulmonary exercise gas exchange analyzer that gathers the observations to be classified or described, b) a feature extraction mechanism that computes numeric information from the observations, and c) a classification or description scheme that does the actual job of classifying or describing observations based on the extracted features.

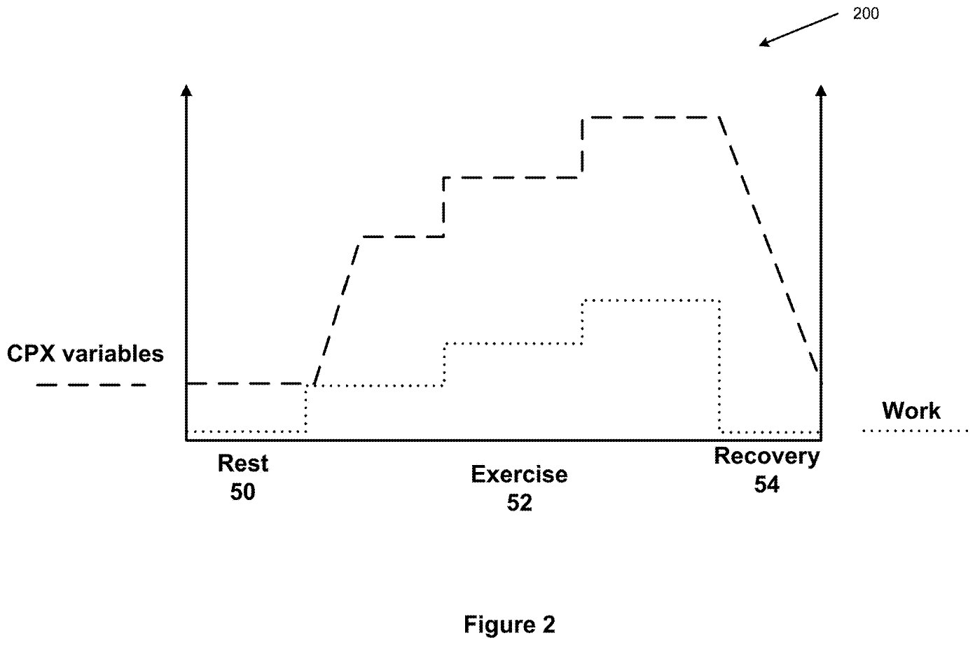

Data Gathering: As indicated and shown in graph 200 of FIG. 2, the general class of data utilized in the present disclosure, cardiopulmonary exercise gas exchange measurements, is obtained 1) at rest, 2) during physical exercise testing performed in accordance with a standardized incremental workload protocol as the forcing function to elicit physiologic changes resulting from the increasing workload, and 3) during a short recovery period following exercise termination. The data measured during exercise quantifies how an individual is able to function in the physical world in terms of the physiologic changes that the individual experiences when engaged in the performance of daily physical work.

The physiologic changes are measured using a CPX testing system to measure selected variables associated with one or more of oxygen consumption, VO₂, carbon dioxide production, VCO₂, end tidal ETCO₂, mixed expired CO₂, PECO₂, HR, and SpO₂.

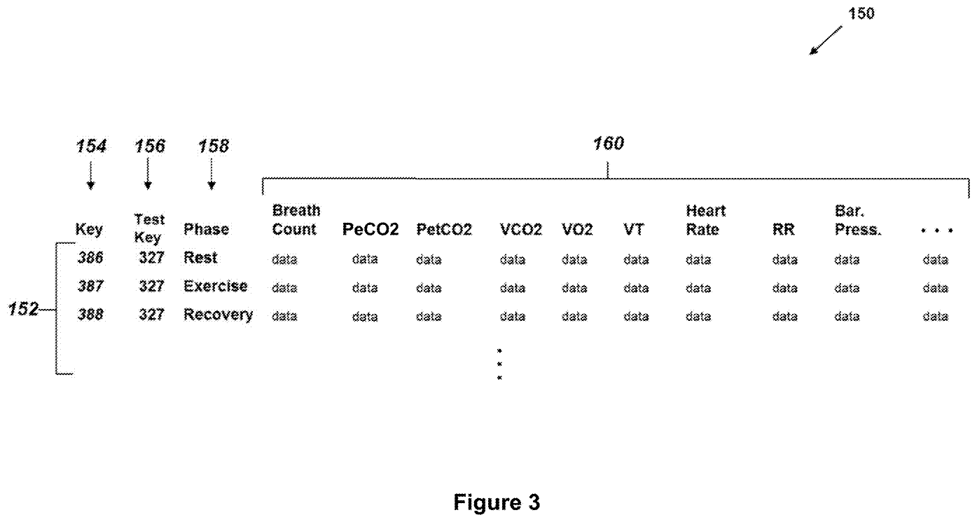

As indicated, the data gathering aspect of the disclosure involves known techniques and analyses, and the calculations for formulating predictive assessments are readily available in the scientific literature (see the bibliography in References). However, by means of aspects of the feature extraction mechanism, classification and quantification scheme, the present disclosure enables an observer to gain new and valuable insight into the present condition and condition trends in patients. Thus, in accordance with a preferred method, a cardiopulmonary exercise gas exchange analysis is made for each test data set (FIG. 3). The performance of such a test is well understood by individuals skilled in the art, and no further explanation of this is believed necessary.

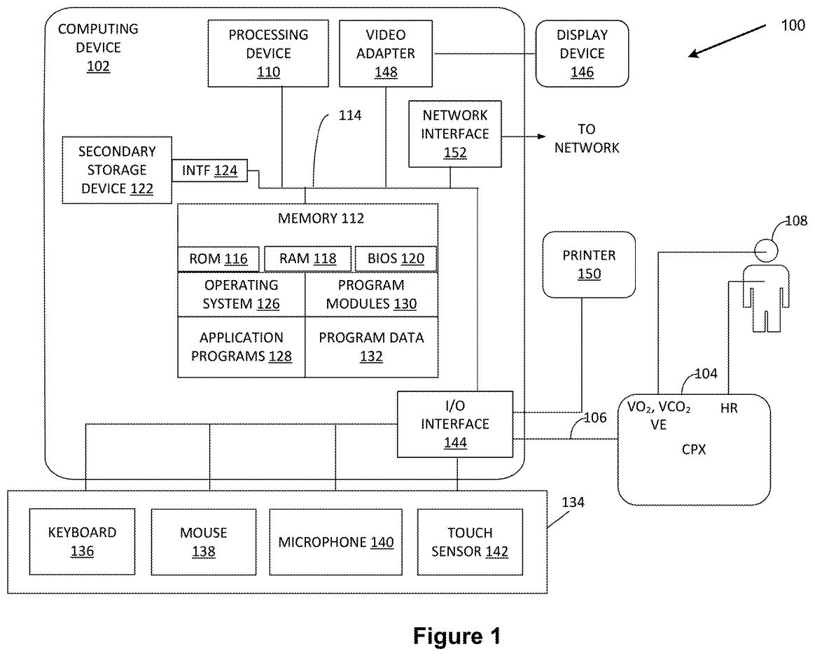

Equipment—With this in mind, an example system is shown in FIG. 1, which illustrates an example CPX testing system 100, whereby a CPX test may be conducted and the results displayed in accordance with the method of the present disclosure. In this example, the system includes a computing device 102. The computing device 102 illustrated in FIG. 1 can be used to execute the operating system, application programs, and software modules described herein.

The computing device 102 includes, in some embodiments, at least one processing device 110, such as a central processing unit (CPU). A variety of processing devices are available from a variety of manufacturers, for example, Intel or Advanced Micro Devices. In this example, the computing device 102 also includes a system memory 112, and a system bus 114 that couples various system components including the system memory 112 to the processing device 110. The system bus 114 is one of any number of types of bus structures including a memory bus, or memory controller; a peripheral bus; and a local bus using any of a variety of bus architectures.

Examples of computing devices suitable for the computing device 102 include a desktop computer, a laptop computer, a tablet computer, a mobile computing device (such as a smart phone, an iPod® or iPad® mobile digital device, or other mobile devices), or other devices configured to process digital instructions.

The system memory 112 includes read only memory 116 and random access memory 118. A basic input/output system 120 containing the basic routines that act to transfer information within computing device 102, such as during start up, is typically stored in the read only memory 116.

The computing device 102 also includes a secondary storage device 122 in some embodiments, such as a hard disk drive, for storing digital data. The secondary storage device 122 is connected to the system bus 114 by a secondary storage interface 124. The secondary storage devices 122 and their associated computer readable media provide nonvolatile storage of computer readable instructions (including application programs and program modules), data structures, and other data for the computing device 102.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory computer-readable media. Additionally, such computer readable storage media can include local storage or cloud-based storage.

A number of program modules can be stored in secondary storage device 122 or system memory 112, including an operating system 126, one or more application programs 128, other program modules 130 (such as the software engines described herein), and program data 132. The computing device 102 can utilize any suitable operating system, such as Microsoft Windows™, Google Chrome™ OS or Android, Apple OS, Unix, or Linux and variants and any other operating system suitable for a computing device. Other examples can include Microsoft, Google, or Apple operating systems, or any other suitable operating system used in tablet computing devices.

In some embodiments, a user provides inputs to the computing device 102 through one or more input devices 134. Examples of input devices 134 include a keyboard 136, mouse 138, microphone 140, and touch sensor 142 (such as a touchpad or touch sensitive display). Other embodiments include other input devices 134. The input devices are often connected to the processing device 110 through an input/output interface 144 that is coupled to the system bus 114. These input devices 134 can be connected by any number of input/output interfaces, such as a parallel port, serial port, game port, or a universal serial bus. Wireless communication between input devices and the interface 144 is possible as well, and includes infrared, BLUETOOTH® wireless technology, 802.11a/b/g/n, cellular, ultra-wideband (UWB), ZigBee, or other radio frequency communication systems in some possible embodiments.

In this example embodiment, a display device 146, such as a monitor, liquid crystal display device, projector, or touch sensitive display device, is also connected to the system bus 114 via an interface, such as a video adapter 148. In addition to the display device 146, the computing device 102 can include various other peripheral devices, such as a printer 150 for printing reports or speakers (not shown).

When used in a local area networking environment or a wide area networking environment (such as the Internet), the computing device 102 is typically connected to the network through a network interface 152, such as an Ethernet interface or WiFi interface. Other possible embodiments use other communication devices. For example, some embodiments of the computing device 102 include a modem for communicating across the network.

The computing device 102 typically includes at least some form of computer readable media. Computer readable media includes any available media that can be accessed by the computing device 102. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 102.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device 102 illustrated in FIG. 1 is also an example of programmable electronics, which may include one or more such computing devices, and when multiple computing devices are included, such computing devices can be coupled together with a suitable data communication network so as to collectively perform the various functions, methods, or operations disclosed herein.

The equipment used in the exercise protocol can be a simple stair step of a known height or any other exercise modality such as a treadmill, bike, or hand ergometer. A CPX testing system 104 interfaces with the subject 108 (the subject is also referred to as a patient) during operation of the exercise test. The physiological variables may be selected from heart rate (HR), ventilation (VE), rate of oxygen uptake or consumption (VO₂) and carbon dioxide production (VCO₂), end tidal CO₂ (PetCO₂), mixed expired CO₂ (PECO₂), or other variables derived from these basic measurements. Physiological data collected is fed into the computing module 102 via a conductor 106, or other communication device.

In some implementations, the CPX system 104 includes a carbon dioxide sensor that can determine a concentration of carbon dioxide in a gas sample. Some implementations also include an oxygen sensor that can determine a concentration of oxygen in a gas sample. Some implementations do not include an oxygen sensor. The CPX system 104 may also include a flow volume sensor that can determine a flow volume of gas exhaled or inhaled by the patient. For example, the flow volume sensor may include one or more pressure transducers. The pressure transducers may determine a change in pressure as the gas passes through an orifice in tube through which the patient is breathing. Based on the change in pressure, the flow volume may be determined for the gas being exhaled and inhaled by the patient. These flow volume measurements may be combined with one or more of the CO₂ or O₂ measurements from the corresponding sensors to determine various parameters of the patient as described further herein.

The workload protocol is illustrated in FIG. 2 and is organized into a rest phase 50, and exercise phase 52, and a recovery phase 54. Although not required, the workload may also be quantified, if a step is used as the exercise modality, by requiring the patient to maintain a desired stepping cadence by the addition of an audible metronome that guides the frequency of the steps taken during the exercise phase. Other prompts may be used to quantify or encourage patients to achieve or maintain a desired workload with other exercise modalities.

Data acquired by the CPX testing system may be stored in a relational database or another data store as illustrated in FIG. 3. Most importantly, data for each patient and each test is stored into separate subsets of data representing the rest phase 386, the exercise phase 387, and the recovery phase 388 for use by the feature extraction mechanism.

An advantage provided by at least some embodiments of the present disclosure is that many of the most prevalent chronic diseases can be identified without the measurement of oxygen consumption. The significance of this is that the most expensive and problematic sensor traditionally used in a CPX system can be excluded from the equipment shown in FIG. 1. Also, the most reliable O2 sensor is large, vibration-sensitive and bulky, while the differential pressure transducer (air flow) and CO₂ sensor are fingernail sized.

Some implementations include a gas analyzer that is an oxygen-sensorless analyzer. An oxygen-sensorless analyzer is a gas analyzer that does not include an oxygen sensor. For example, an oxygen-sensorless analyzer may include a differential pressure transducer and a carbon dioxide sensor but not an oxygen sensor.

An example embodiment includes a wearable equipment package that includes a differential pressure transducer and a CO₂ sensor and employs technology to communicate with a mobile computing device, such as a smart phone, rather than a personal computer such as a desktop personal computer (PC) or laptop PC. In some embodiments, the wearable equipment package includes an O₂ sensor. In some embodiments, the wearable package does not include an O₂ sensor. In some implementations, the mobile computing device may use an application (or app) that is installed and specially programmed to communicate with the wearable device over a wireless or wired protocol. The wearable equipment package could be used in a disease screening tool in primary care clinics. Whether a PC is used or a smart phone app is used, the outputs may be similar to those described further herein.

### Feature Extraction Steps

Some implementations use the steps described below for feature extraction. Some implementations do not include all steps, perform the listed steps in an alternate order, or include different steps or variations of the steps listed below.

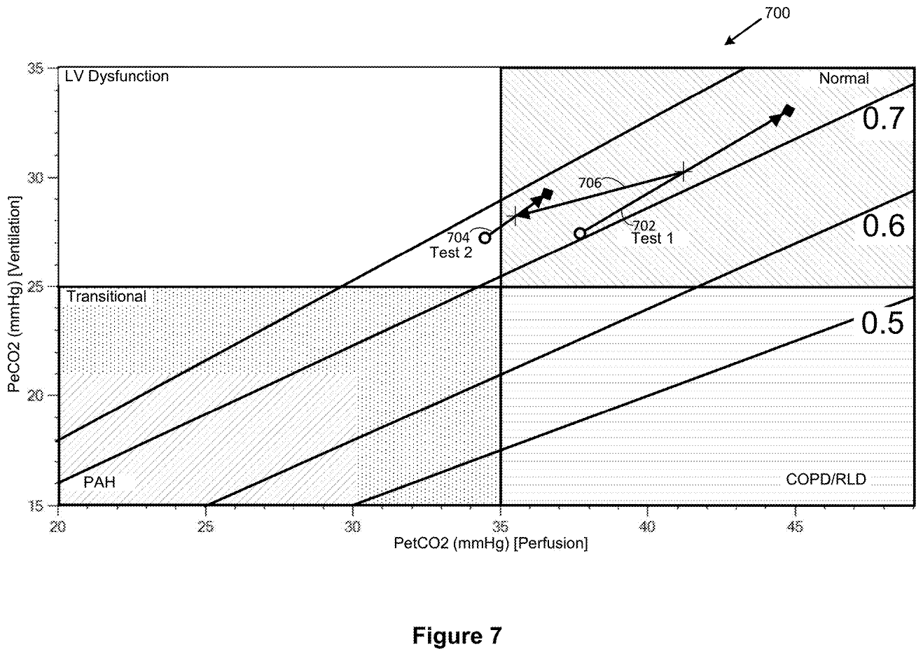

Step 1—Detection—In some implementations, the fixed format plot 600 illustrated in FIG. 6 is first populated with the CPX testing system measured variables mixed expired CO₂ (PECO₂) as the y value and end tidal CO₂ (PetCO₂) on the x axis, which are both plotted in mmHG in this example. These measurements may be used to define a vector for a test, as shown in FIG. 7. A vector 702 for Test 1 is shown, with one end at the resting value, indicated by an open circle, and one end on the end exercise value, indicated by a square box. An arrow symbol may then be added next to the square box to further indicate direction and amount of change from rest to exercise. A midpoint may be identified somewhere between the first end and the second end. The midpoint may be the geometric mean (i.e., the median x,y value) of the first end and the second end. In other words, the midpoint may be the half-way point between the first end and the second end of the test vector. In some implementations, other methods of determining a midpoint along the vector are used. The midpoint may be computed as any point along the vector. In this example, the midpoint is the geometric mean and is computed and identified on the plot using a + symbol. In this manner, the interpreting physician can easily identify the most likely disease type by observing the zone (or quadrant) in which the median of the vector (+ symbol) is located.

Step 2—Trend Plots—For patients who have had more than one CPX test (e.g., tests on more than one day), one vector may be plotted for each test. Referring to FIG. 7, two such vectors are shown (one per unique test), each with its own median value, are shown on a plot 700. In addition to the vector 702, FIG. 7 also include a vector 704. The test sequence is displayed—from the first test to the second test. An additional vector 706 is drawn from the median of the first vector to the median point of the second vector. In the example of FIG. 7, the resultant vector 706 of the medians is pointing left and down. This shows the interpreting physician that this patient's suspected disease is worsening or is not responding well to therapy, because the direction of the vector of the medians is proceeding from the Normal quadrant towards the Transitional quadrant. For a positive therapy responding patient, this vector should proceed upward and to the right indicating improvement in both lung ventilation and perfusion.

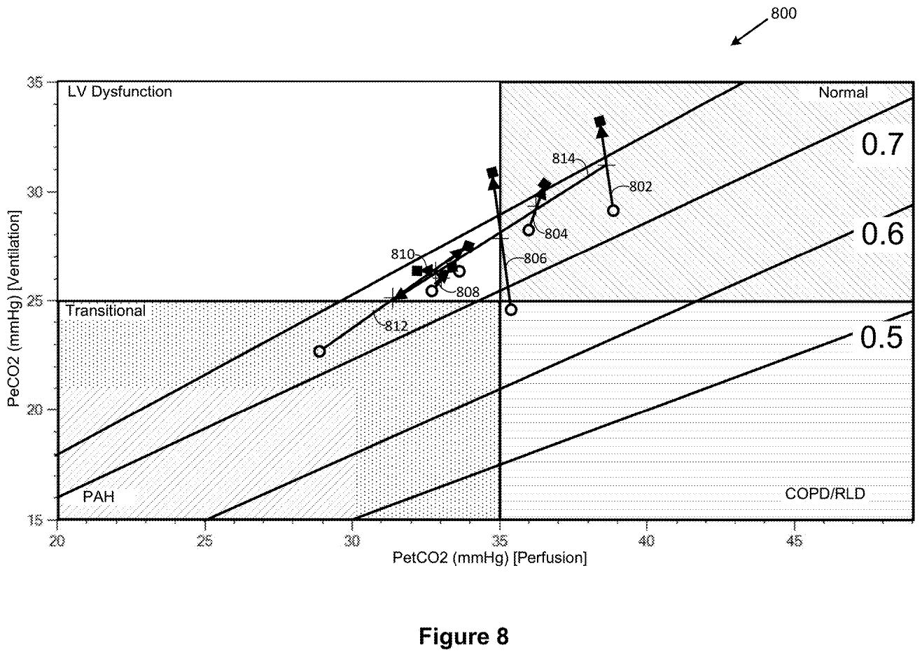

Referring to FIG. 8, a plot 800 for a patient with multiple CPX tests (a total of 6 in this example, represented by vectors 802, 804, 806, 808, 810, and 812) can become very confusing. Additionally, in this example, a vector 814 is shown between the midpoint of the vector 802 (representing the earliest in time test for the patient) and the vector 812 (representing the latest in time test for the patient). Determining whether a patient is improving with therapy when more than two tests have been performed may be difficult. To preserve the intent of performing multiple tests, namely to determine if a patient is improving with therapy or not, in some implementations only the vectors associated with a subset of the patient's tests are presented. For example, a vector associated with an earlier in time (e.g., a first in time test performed by the patient) and a vector associated with a later in time (e.g., the last in time (or most recent) test performed by the patient) may be selected for display. In some implementations, the first vector may be selected based on the occurrence of an event such as the beginning of a treatment or a change in a treatment. Additionally, in some implementations, a vector of the means of these two tests corresponding to the selected vectors will be displayed similarly to FIG. 7.

The angle of the vector of the medians can also provide objective evidence of the effectiveness of a treatment. Referring to FIG. 7, the angle of a vector of the medians is determined by drawing a vertical (constant PetCO₂) line through the median of the first test. An angle can then be measured between the vertical line and the vector of the medians (i.e., the angle can be measured between the vector of the medians and a vertical vector pointing up).

If the angle is 0 to +90 degrees: Increased ventilation and perfusion post intervention shows the treatment had a positive effect.

If the angle is +90 to +180 degrees: Increased perfusion but decreased ventilation post intervention shows the treatment had mixed success.

If the angle is 0 to −90 degrees: Increased ventilation but decreased perfusion post intervention shows the treatment had mixed positive effect.

If the angle is −90 to −180 degrees: Decreased ventilation and decreased perfusion post intervention shows the treatment had no positive success.

This testing strategy will also work for fast acting therapies, such as a beta 2 agonist bronchodilator for either exercise induced asthma or for ameliorating hyperinflation during exercise in COPD patients. Because the CPX test is short (6 minutes), a first test is performed, after which the patient rests and a bronchodilator is given. Shortly thereafter, a second test is initiated. This would produce two vectors, the medians of which are used to generate a vector of the medians similarly to that described above. If this vector is pointing up and to the right (angle of the medians of the vectors >=0 and <=90), the bronchodilator provided an indication of reduced exercise induced bronchospasm with improved lung ventilation and perfusion.

Based on the determined physiological condition of a patient or the determined change in the condition of the patient based on the tests, one or more known treatment options for the condition or observed change in condition may be presented. A physician or physician may then select an appropriate option or determine an appropriate treatment or therapy change. An example therapy option for PAH could include the physician prescribing an endothelin receptor blocker (ERB) to reduce pulmonary vascular resistance and improve pulmonary blood flow or increase PETCO2 during rest and exercise.

### Classification Scheme

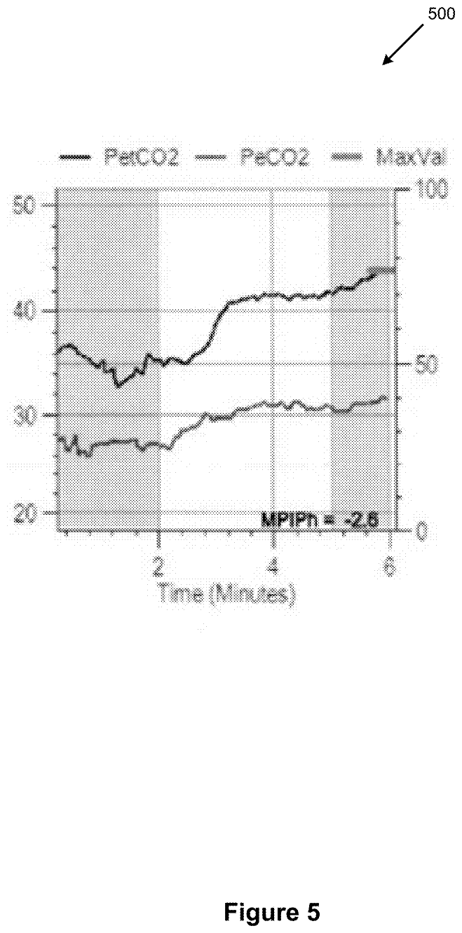

The primary description scheme is the design of the ventilation/perfusion plot as in FIG. 6 itself, which shows 4 quadrants for normal, COPD, and PAH. The lower left quadrant is further divided into a PAH zone and a Transition zone. These zones could be determined by visual observation of the graph 500 shown in FIG. 5. However, a further discussion is important to understand the meaning of each zone and the physiologic context of each:

Normal zone—Patients who fall into this zone based on the mean value of the vector described above have normal ventilation and perfusion changes with exercise. This zone is the target for any and all forms of therapy—a return to normal.

COPD—Patients who fall into this zone based on the mean value of the vector described above have slightly reduced perfusion but have clearly limited ventilation due to their lung disease, whether it be obstructive or restrictive.

LV dysfunction zone—The LV dysfunction zone reflects upon patients who demonstrate a reduction in pulmonary blood flow or cardiac output due to left ventricular pump dysfunction, whether it be systolic or diastolic. The reduced pulmonary blood flow is reflected by a blunting in the rise of PETCO2 during exercise. Regarding changes or increases in ventilation in patients with LV dysfunction, there is normally still an increase in PECO₂ or mixed expired CO₂ during exercise, unless the patient has co-morbid lung disease aside from their LV dysfunction.

PAH and Transitional-Patients whose median of the test vector falls into one of these zones have both defective perfusion and ventilation due to their pulmonary vascular restrictions. The zone identified as Transitional, however, means the patient may not be classified as a PAH patient, which has a poor prognosis, nor can the patient be classified as a COPD patient. Thus, they are in transition and progressing to increased disease severity. This transition zone shows evidence of reduced perfusion (lower PETCO₂ than normal or early LV dysfunction patient profile) and reduced ventilation (PECO₂), similar to the profile for patients with an early developing COPD profile. A patient test that produces a test vector median in the Transitional Zone is undergoing change, possible as a result of therapy. However, it could also be caused by the process of pulmonary vascular disease progression. This is where another CPX variable such as GxCap, a correlate of pulmonary capacitance, is helpful to show that the disease process progresses (reduced PetCO₂) until Pulmonary Vascular Resistance starts to increase, thereby placing a load on the Right Ventricle and further reducing blood flow and stroke volume (O₂P). The lungs can respond with bronchoconstriction (reduced PECO₂ and ventilation) or arteriolar vascular constriction to favor a more balanced V/Q ratio. As RV function deteriorates, capacitance, or GxCap, further decreases thus reducing cardiac output. Thus, the Transitional Zone is the early detection zone for suspected PAH. This may be shown, as in FIGS. 7 and 8, by the direction of the vector of the means (described above) proceeding downward and to the left.

To enhance the primary quadrant selection, such as with LV dysfunction, variables from other supportive diagnostic procedures (LV EF, mean PA and PCW pressure) may be displayed providing supportive evidence of worsening LV dysfunction. Worsening LV dysfunction can shift the vector of the medians into the transitional zone towards the PAH zone, indicating worsening RV disease progression. Other descriptive PAH metrics such as GxCap, a strong surrogate to pulmonary artery capacitance determined by hemodynamics, can be displayed in the V/Q plot or elsewhere to provide additional supportive evidence for a transition quadrant vector moving to the PAH zone. In some implementations, impressions statements may be generated based on GxCap or a GxCap recovery vector, which is discussed further below, if it is determined that the median or an endpoint of a patient's test vector falls into the PAH zone. Additionally, impressions statements may be generated based on the recovery vector when the test vector falls within the PAH zone. For example, an impressions statement may include text that indicates that the GxCap recovery vector (or the directional change during recovery of GxCap) is supportive evidence of PAH.

In some implementations, a recovery vector is determined and displayed. The recovery vector may be determined based on changes in various physiological measures that occur between the beginning and ending of a recovery phase of a test. In some implementations, the recovery vector is determined based on comparing a value determined during an exercise phase with a value determined during a recovery phase. The recovery vector may be based, for example, on GxCap. In some implementations, the recovery vector may be used for vaso-activity assessment.

Vaso-activity assessment may include pulmonary vascular function or relaxation testing and may be referred to as pulmonary vascular response to recovery testing. If the pulmonary vasculature responds favorably to an intervention, such as intravenous epoprostenol or adenosine or nitric oxide inhalation, a greater increase in the dynamic vector mid-point value (longer recovery segment of the vector shifted to the right) during the recovery period or a greater delta from end exercise to end recovery descriptive of drug induced vaso-relaxation may be observed. In some implementations, PeCO₂ changes are plotted on the y-axis against PetCO₂ changes on the x-axis. An improvement in PetCO₂ with use of a vaso-dilator drug during a test will extend the recovery segment and shift the vector downward, as seen in patients with normal pulmonary-vascular function.

Some implementations use the recovery vector to support or infer the presence of COPD based at least in part on detecting little change from end exercise to end recovery in the PeCO₂/PetCO₂ ratio due to a lower PeCO₂ value relative to PetCO₂.

Some implementations may include a plot of PeCO₂ and PetCO₂ with time. A drug that causes vaso relaxation such as those mentioned above as examples but not limited to such medications, may result in a test that shows an increase in the PetCO₂ immediately following drug administration, which may also be reflected in a more rightward end of recovery point in the V/Q plot.

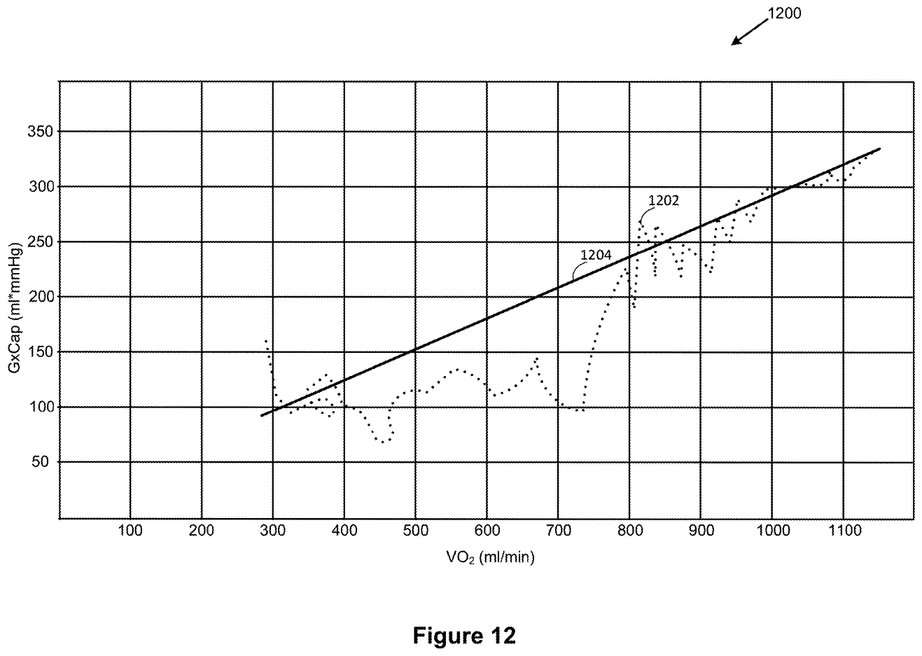

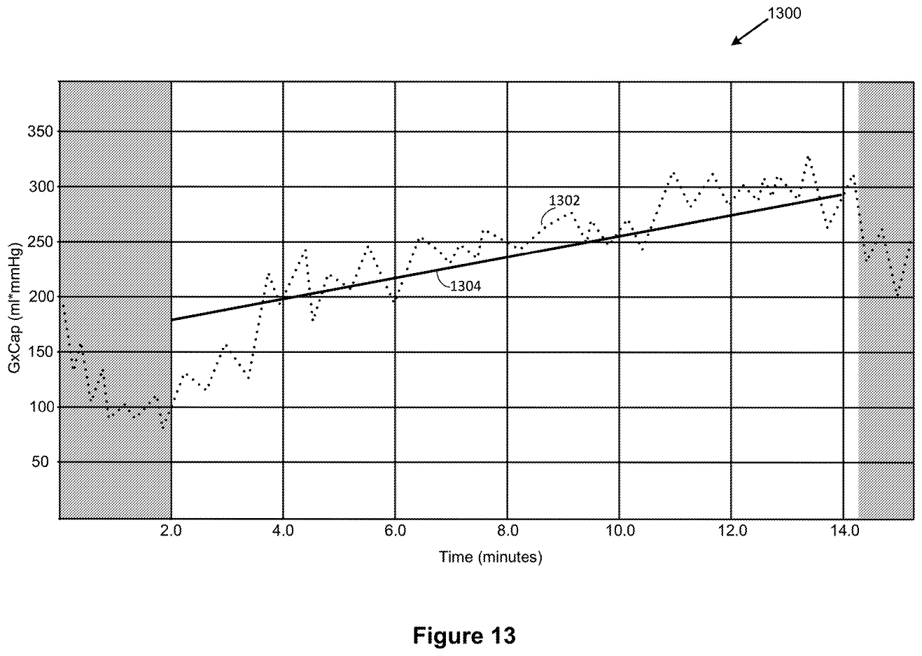

GxCap may also be displayed on another plot. For example, GxCap may be plotted against time as shown in FIG. 12 or VO₂ as shown in FIG. 13. FIG. 12 shows a plot 1200 having a data series 1202 with GxCap values for the y-coordinate and VO₂ values for the x-coordinate. The values in the data series 1202 may be collected during a test. A regression line 1204 is also shown on the plot 1200. The regression line 1204 may be generated from some or all of the values in the data series 1202. A linear slope may be determined from the regression line 1204. FIG. 13 shows a plot 1300 having a data series 1302 with GxCap values for the y-coordinate and time values for the x-coordinate. The values in the data series 1302 may be collected during a test. For example, the data series 1302 may be from the same test used to generate the data series 1202. A regression line 1304 is also shown on the plot 1300. The regression line 1304 may be generated from some or all of the values in the data series 1302. A linear slope may be determined from the regression line 1304. Some implementations may use one or both of the slope of the regression line 1204 and the slope of the regression line 1304 for vaso-activity assessment or classifying a physiological condition of a patient. In some implementations, the plot 1200 or the plot 1300 may be generated for a CPX test based on determining that a midpoint or endpoint of the test vector is within the PAH zone of a plot (e.g., of the plot 600 shown in FIG. 6). For example, a user interface that shows the plot 1200 or the plot 1300 may automatically be displayed based on determining the test vector (or a portion of the test vector) is within the PAH or transitional zone.

### Impressions Statement

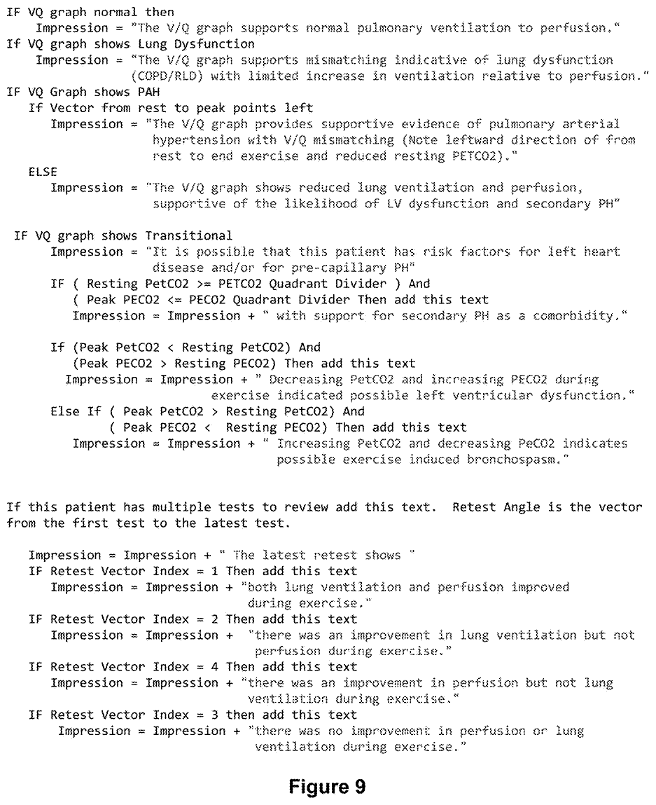

Since most commercial providers of medical diagnostic equipment cannot “interpret” a test (a license to practice medicine is needed to interpret a test), the present method uses the term “Impressions” to verbalize the key findings of a CPX test. The physician, upon review of the test results, will either keep the computer-generated text as is, or he/she will edit the impressions statement. This is especially valuable to make sense out of complicated tests that fall into the Transition zone. This logic of this statement can also include other CPX variables collected during CPX testing. FIG. 9 shows example logic used to render an impressions statement in at least some implementations.

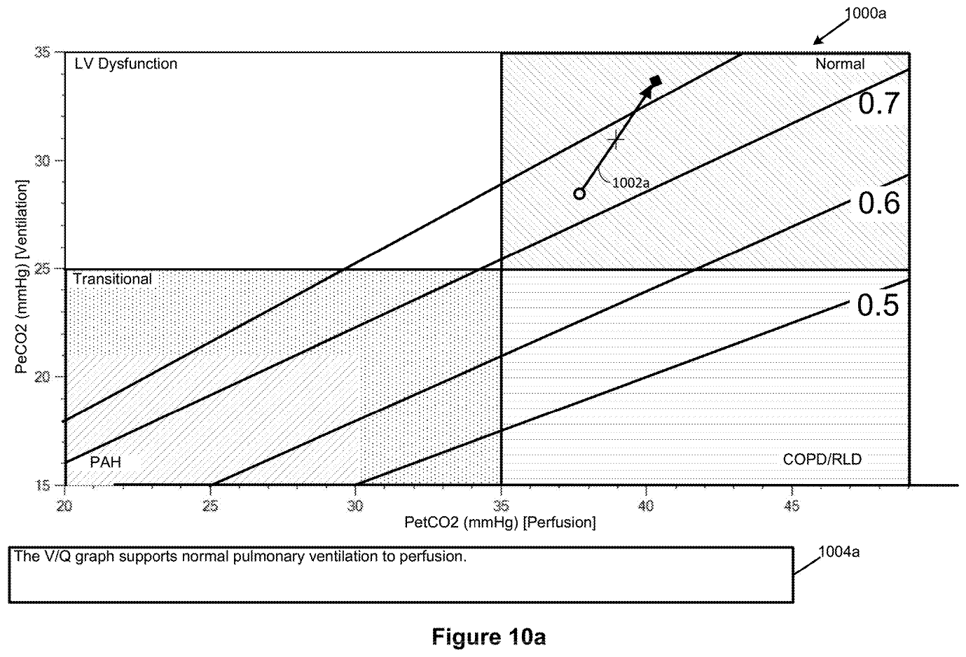

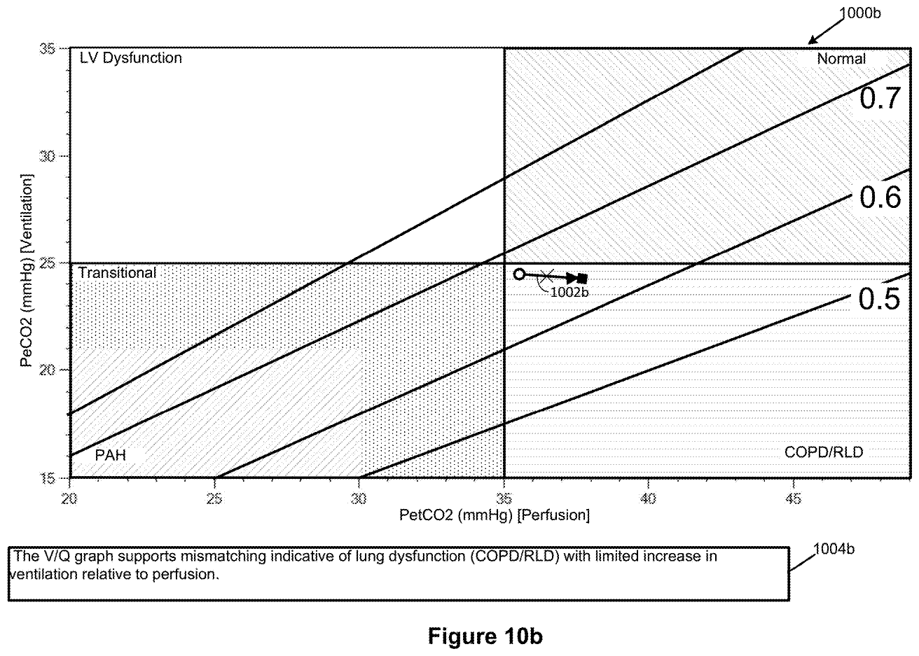

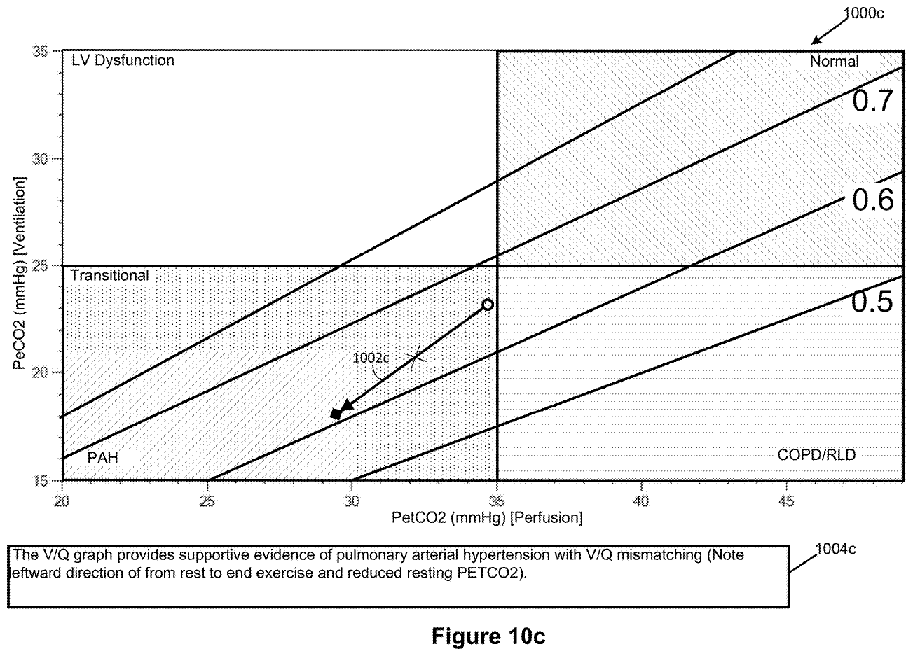

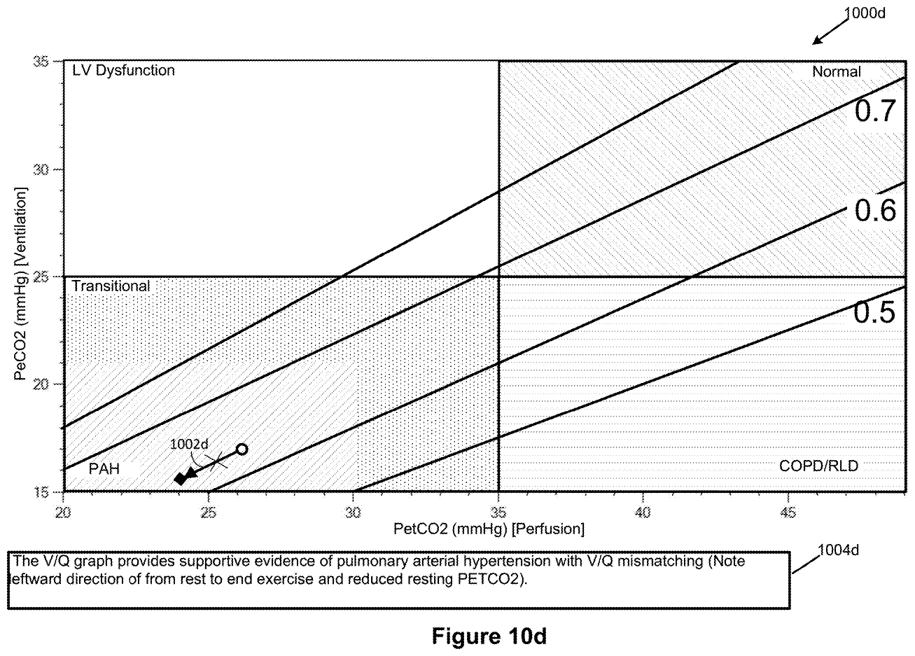

FIGS. 10a-d are provided to illustrate how example test reports would appear for four physiological conditions or disease states. FIG. 10a shows an example test report with a vector 1002a from a test with results in the normal zone. FIG. 10a also shows example impressions 1004a generated based on the test. FIG. 10b shows an example test report from a test with results in the COPD zone and example impressions 1004b generated based on the test. FIG. 10c shows an example test report with a vector 1002c from a test with results primarily in the transitional zone and example impressions 1004c generated based on the test. FIG. 10d shows an example test report with a vector 1002d from a test with results in the PAH zone and example impressions 1004d generated based on the test.

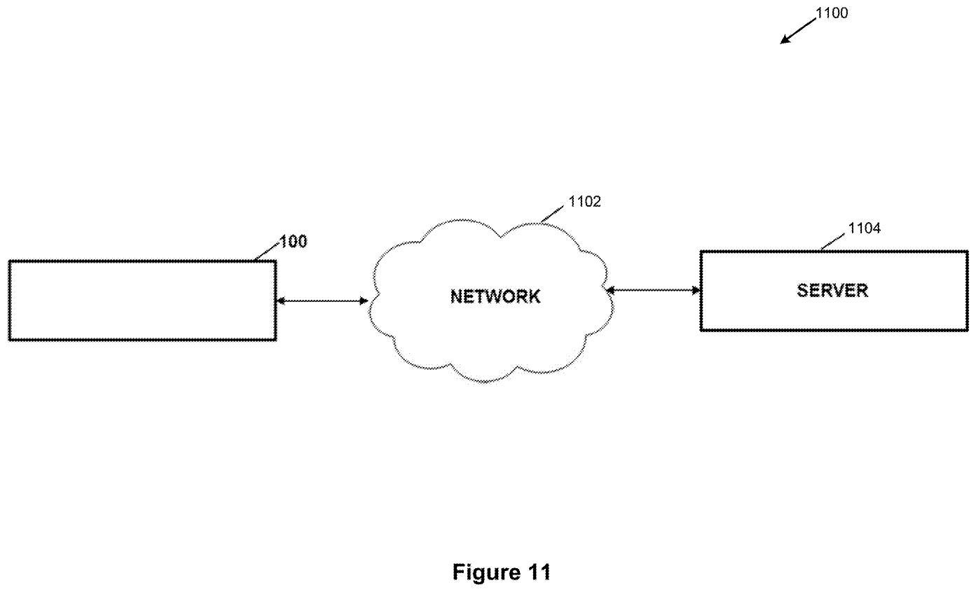

Referring now to FIG. 11, an exemplary networked CPX testing system 1100 is illustrated. The networked CPX testing system 1100 can be used for remote testing and monitoring of patients. For example, the networked CPX testing system 1100 can be used in non-clinical environments, such as at the patient's home. In this example, the networked CPX testing system 1100 includes the CPX testing system 100, network 1102, and server 1104.

In some embodiments, the CPX testing system 100 is configured to send data associated with gas exchange tests (such as measurements of physiological parameters, index scores, etc.) to the server 1104 over the network 1102.

The network 1102 is an electronic communication network that facilitates communication between the CPX testing system 100 and the server 1104. An electronic communication network is a set of computing devices and links between the computing devices. The computing devices in the network use the links to enable communication among the computing devices in the network. The network 1102 can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices.

In various embodiments, the network 1102 includes various types of links. For example, the network 1102 can include wired and/or wireless links, including Bluetooth, ultra-wideband (UWB), 802.11, ZigBee, and other types of wireless links. Furthermore, in various embodiments, the network 1102 is implemented at various scales. For example, the network 1102 can be implemented as one or more local area networks (LANs), metropolitan area networks, subnets, wide area networks (such as the Internet), or can be implemented at another scale.

The server 1104 comprises one or more computing devices. Various embodiments of computing devices have been described above. Further, in some embodiments, the server 1104 comprises a single server or a bank of servers. In another example, the server 1104 can be a distributed network server, commonly referred to as a “cloud” server.

In some embodiments, the server 1104 operates to receive data such as test results and physiological measurements from the CPX testing system 100. The server 1104 can then process the data and store it in one or more of a database or electronic medical records system.

In some embodiments, the server 1104 generates user interfaces, such as with a user interface engine, and transmits those user interfaces for display remotely. For example, the server 1104 may generate a web page comprising a user interface containing test data transmitted from the CPX testing system 100. The web page may then be transmitted to a computing device (e.g., a smart phone, personal computer, or tablet) of the patient or a physician.

Additionally, in some embodiments, the CPX testing system 100 communicates with a cellular phone or other network-connected computing device to access the network 1102. For example, the CPX testing system 100 may transmit data to the server 1104 via communication with a cell phone using Bluetooth. Other embodiments are possible as well.

The embodiments disclosed herein may be used in a scoring system based on several CPX test variables that are used to determine the height of “disease silos” graphic representations of the likeliness that the cause of a patient's dyspnea is one or more of the represented the disease silos. For example, embodiments may be incorporated into a system for determining and displaying “disease silos” such as those described in U.S. Pat. No. 10,010,264, titled “Pattern recognition system for quantifying the likelihood of the contribution of multiple possible forms of chronic disease to patient reported dyspnea” and dated Jul. 3, 2018. This allows a non-expert physician to emulate the thought process of experienced physicians and physiologists to determine the primary and secondary causes of the patient's shortness of breath. The methods described herein may improve the likeliness scoring by expanding the individual silo scoring schemes to include the Ventilation/Perfusion measurements of the present method to the scoring algorithms disclosed in in U.S. Pat. No. 10,010,264.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

In the following several examples are given.

Example 1: A computer-implemented method comprising: receiving first cardiopulmonary exercise test data for a patient; plotting a first vector based on the first cardiopulmonary exercise test data, the first vector including a first point based on a rest value from the first cardiopulmonary exercise test data and a second point based on an exercise value from the first cardiopulmonary exercise test data, a first coordinate value of the first point and a first coordinate value of the second point being based on mixed expired CO2 (PECO2) and a second coordinate value of the first point and a second coordinate value of the second point being based on end tidal CO2 (PetCO2); and triggering display of the plotted vector over a coordinate grid.

Example 2: The computer-implemented method of example 1, wherein the plotting a first vector includes plotting an arrow that begins at the first point and ends at the second point.

Example 3: The computer-implemented method of example 1, further comprising: receiving second cardiopulmonary exercise test data for the patient, the first cardiopulmonary exercise test data corresponding to a first cardiopulmonary exercise test performed by the patient and the second cardiopulmonary exercise test data corresponding to a second cardiopulmonary exercise test performed by the patient; and plotting a second vector based on the second cardiopulmonary exercise test data.

Example 4: The computer-implemented method of example 3, wherein the receiving first cardiopulmonary exercise test data for the patient includes selecting cardiopulmonary test data corresponding to an earlier in time cardiopulmonary exercise test performed by the patient and the receiving second cardiopulmonary exercise test data for the patient includes selecting cardiopulmonary test data corresponding to a later in time cardiopulmonary test performed by the patient.

Example 5: The computer-implemented method of example 3, further comprising classifying the effectiveness of a treatment based on the first vector and the second vector.

Example 6: The computer-implemented method of example 5, wherein the classifying the effectiveness of the treatment based on the first vector and the second vector includes comparing a midpoint of the first vector to a midpoint of the second vector.

Example 7: The computer-implemented method of example 6, wherein the midpoint of the first vector is the geometric mean of the first point and the second point.

Example 8: The computer-implemented method of example 3, further comprising plotting a third vector, the third vector including a first point based on a midpoint of the first vector and a second point based on a midpoint of the second vector.

Example 9: The computer-implemented method of example 8, further comprising: determining a slope of the third vector; and classifying the effectiveness of a treatment based on the slope.

Example 10: The computer-implemented method of example 9, further comprising generating impressions statements based on the classification of the effectiveness of the treatment.

Example 11: The computer-implemented method of example 10, wherein the impressions statements include a recommendation or supportive information regarding continuation or change of treatment.

Example 12: The computer-implemented method of example 1, wherein the coordinate grid includes a plurality of physiological condition zones.

Example 13: The computer-implemented method of example 12, wherein the plurality of physiological condition zones includes a normal zone, a chronic obstructive and restrictive lung disease zone (COPD), a left ventricular (LV) dysfunction zone, a pulmonary arterial hypertension (PAH) zone, and a transitional zone.

Example 14: The computer-implemented method of example 1, further comprising determining a physiological condition classification for the patient based on the plotted vector.

Example 15: The computer-implemented method of example 14, further comprising generating impressions statements based on the physiological condition classification.

Example 16: The computer-implemented method of example 1, further comprising: plotting a midpoint of the first vector; and triggering display of the plotted midpoint over a coordinate grid having a plurality of physiological condition zones.

Example 17: A non-transitory computer-readable storage medium comprising instructions stored thereon that, when executed by at least one processor, are configured to cause a computing system to: receive first cardiopulmonary exercise test data for a patient; plot a first vector based on the first cardiopulmonary exercise test data, the first vector including a first point based on a rest value from the first cardiopulmonary exercise test data and a second point based on an exercise value from the first cardiopulmonary exercise test data, a first coordinate value of the first point and a first coordinate value of the second point being based on mixed expired CO2 (PECO2) and a second coordinate value of the first point and a second coordinate value of the second point being based on end tidal CO2 (PetCO2); and trigger display of the plotted vector over a coordinate grid having a plurality of physiological condition zones.

Example 18: The non-transitory computer-readable storage medium of example 17, wherein the instructions further cause the computing system to classify the patient with respect to a physiologic condition based on the first vector and the physiological condition zones.

Example 19: A non-transitory computer-readable storage medium comprising instructions stored thereon that, when executed by at least one processor, are configured to cause a computing system to: receive cardiopulmonary exercise test data for a patient; and plot a recovery vector based on the first cardiopulmonary exercise test data.

Example 20: The non-transitory computer-readable storage medium of example 19, wherein the recovery vector includes a first recovery vector point based on an end exercise value from the cardiopulmonary exercise test data and a second recovery vector point based on an end recovery value from the cardiopulmonary exercise test data.

Example 21: The non-transitory computer-readable storage medium of example 18, wherein a first coordinate value of the first recovery vector point and a first coordinate value of the second recovery vector point are based on GxCap.

Example 22: The non-transitory computer-readable storage medium of example 18, wherein a first recovery vector point is determined at the end of an exercise phase and a second recovery vector point is determined at the end of a recovery phase.

Example 23: The non-transitory computer-readable storage medium of example 19, wherein the recovery vector is based on a directional change in GxCap versus time.

Example 24: A system comprising: a flow sensor configured to sense a respiratory flow of a patient; an analyzer configured to determine a composition of at least a portion of the respiratory flow of the patient; and a computing device configured to: receive first cardiopulmonary exercise test data for a patient; plot a first vector based on the first cardiopulmonary exercise test data, the first vector including a first point based on a rest value from the first cardiopulmonary exercise test data and a second point based on an exercise value from the first cardiopulmonary exercise test data, a first coordinate value of the first point and a first coordinate value of the second point being based on mixed expired CO2 (PECO2) and a second coordinate value of the first point and a second coordinate value of the second point being based on end tidal CO2 (PetCO2); and trigger display of the plotted vector over a coordinate grid having a plurality of physiological condition zones.

Example 25: The system of example 24, wherein the analyzer includes a carbon dioxide (CO2) sensor.

Example 26: The system of example 25, wherein the analyzer includes an oxygen (O2) sensor.

Example 27: The system of example 25, wherein the analyzer does not include an oxygen (O2) sensor.

Example 28: The system of example 24, wherein the analyzer is an oxygen-sensorless analyzer.

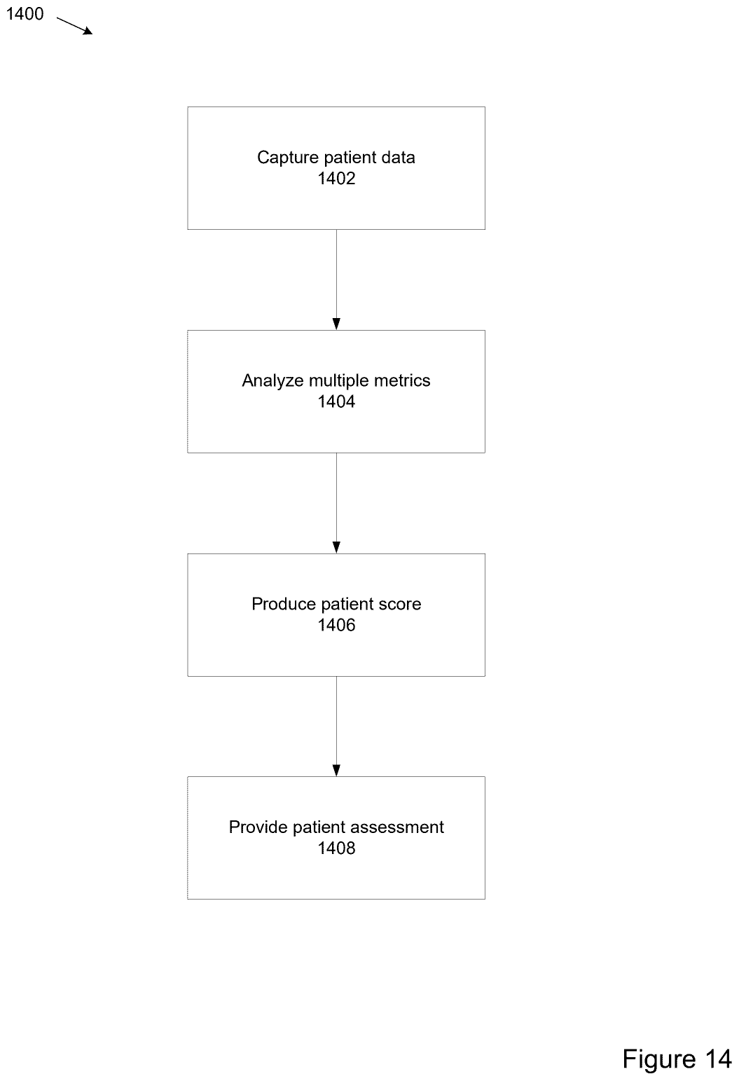

Referring now to FIGS. 14-18, although single exercise gas exchange (Gx) metrics can be useful for determining the degree of functional impairment, such as peak attained VO₂, the bundling of multiple GX based variables has been demonstrated to enhance specific disease detection power and the level of patient risk.

In view of this, the examples provided herein use one or more algorithms based upon Artificial Intelligence (AI) that evaluate “bundled” metrics and variables. These bundled metrics, when evaluated together or combined as a score, can provide more powerful differentiating signatures of heart, lung, or pulmonary vascular disease.

Single Gx variables, like the Minute Ventilation (VE) slope in heart or pulmonary vascular disorders does provide a good tracker variable for disease prognosis. Adding other metrics into a score can enhance disease detection via higher sensitivity and specificity (AUC or area under the curve) or improve therapy monitoring and guidance. such as that with the present method.

One key to augmented diagnosis and especially prognosis is compiling the best variables that provide multiple descriptors of heart, lung and pulmonary vascular function and interaction. In this way, the resolution to optimizing the physician's decisions for best treatment are enhanced. It is believed that the best combination of Gx based variables can improve the accuracy for the physician to determine either the “time to clinical worsening” or improvement to help guide optimal therapy decisions, be it drug up-titration or added medications as a combination therapy.

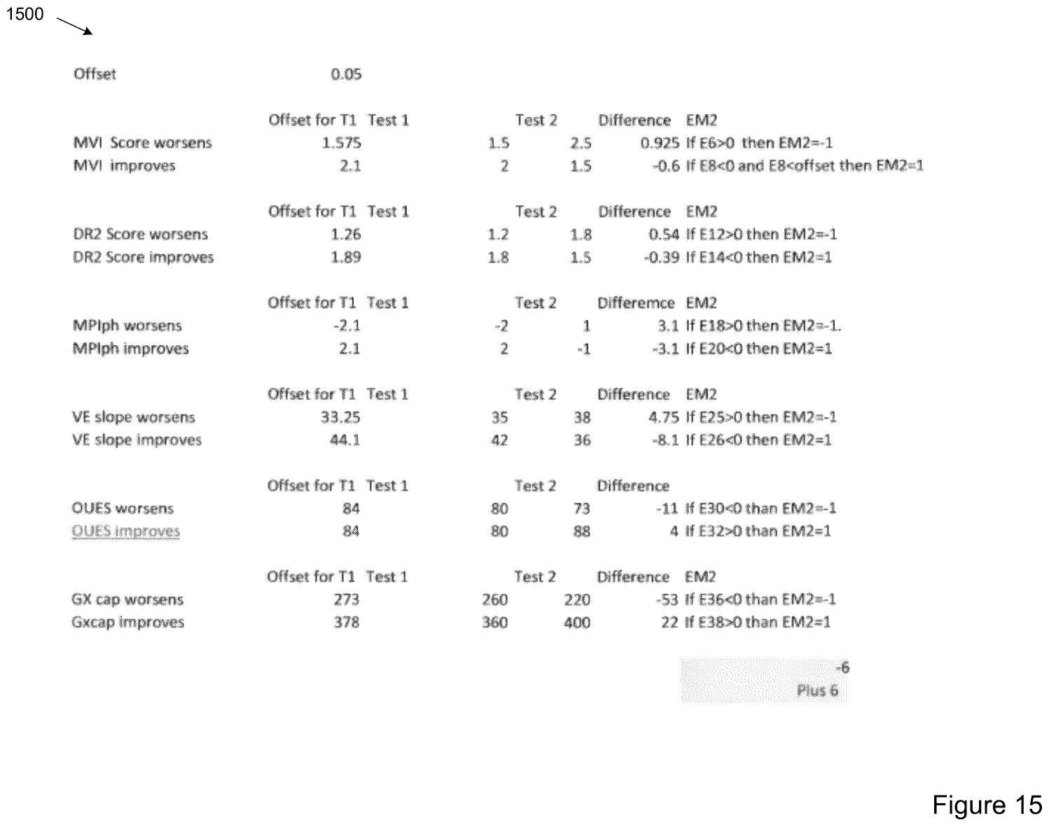

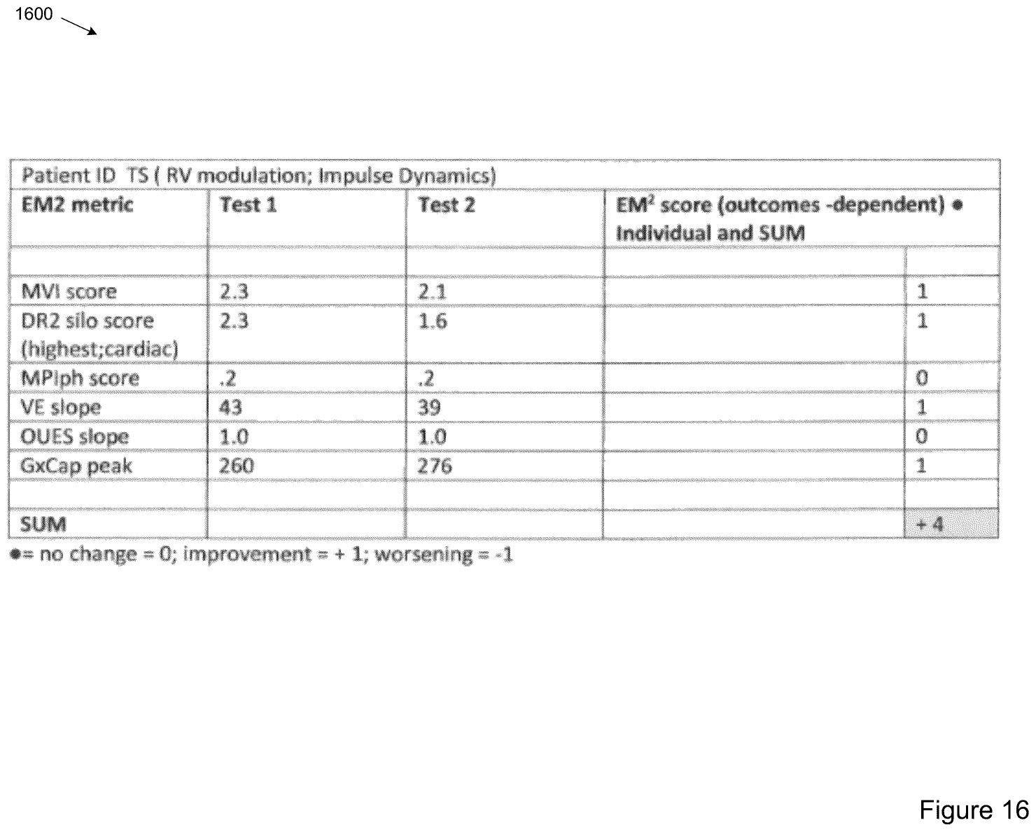

In examples provided herein, up to 6 component variables (see FIGS. 15-16) can be deployed. However, it can be beneficial if just 3 or 4 of the individual parameters change for the positive to confirm patient improvement, not worsening. Stated another way, the best selection of Gx based variables “compressed” into a single score has been proposed to provide a better and more sensitive index for therapy management and monitoring. Assessment of integrative performance of the heart, lungs and pulmonary circulation offers a different and more sensitive assessment of treatment efficacy.

More specifically, there is an unmet physician need for better, higher resolution prognostic tools and guidance for optimal therapy. Such tools can also aid drug companies in their approval trials for drug dose recommendations. Higher resolution graphics and scores can guide drug companies and health care providers as to optimal drugs and most effective doses. The example algorithms provided herein can be used for tracking or assessing drug therapy response. These embodiments can focus on submaximal patient exertion levels and functional “biometrics” using a well-tolerated exercise protocol with simplified test performance and data analysis.

The present disclosure provides a more sensitive, physiologic, and easier to use method than currently available methods intended for the provision of feedback during long-term follow-up and treatment of patients with chronic diseases. In accordance with the present disclosure, a new method has been found for a pattern recognition system that explains gas exchange in the lungs during exercise, including: 1) a cardiopulmonary exercise gas exchange analyzer that gathers the observations to be classified or described, 2) a feature extraction mechanism that computes numeric information from the observations, and 3) a classification or description scheme that does the actual job of classifying or describing observations based on the extracted features.

One of the goals is to eliminate physician “guess work in deciphering directional response to medication selection and dose. Another goal is to provide clear visuals using trending graphics with computed changes in metrics from the current test to the previous one. In addition, impressions statements can be provided to aid physician suggestions of “what to do next”, thereby providing physician guidance and a reduction in physician time for making treatment decisions.

The examples described herein are intended to find the most likely causes of dyspnea during mild to moderate exercise. This improvement is made possible by a) streamlined diagnosis pathways, and b) more accurate algorithms predicting the likelihood of a primary disease and suggesting which other diseases may be present as comorbidities or a second cause of dyspnea. Because there is evidence that this technology detects some of the earliest physiological changes associated with pharmaceutical therapy, for example, it may also enhance earlier diagnosis with a less resource-intensive approach.

In response to this clinical need, an example method 1400 for efficacy management and monitoring (EM2) is provided in FIG. 14.

At optional operation 1402 of the method 1400, patient data is captured. In alternative embodiments, the patient data can be captured by other mechanisms and simply provided to perform the method 1400.

In one example of operation 1402, an incremental submaximal cardiopulmonary exercise testing (CPET) to be used to obtain the data from the patient.

In this example (see, e.g., graph 200 shown in FIG. 2), the general class of data utilized in the present disclosure (cardiopulmonary exercise gas exchange measurements), is obtained: 1) at rest, 2) during physical exercise testing performed in accordance with a standardized incremental workload protocol as the forcing function to elicit physiologic changes resulting from the increasing workload, and 3) during a short recovery period following exercise termination. The data measured during exercise quantifies how an individual is able to function in the physical realm in terms of the physiologic changes that the individual experiences when engaged in the performance of daily physical work.

The physiologic changes are measured using a CPET testing system to measure selected variables associated with one or more of oxygen consumption, VO2, carbon dioxide production (VCO2), end tidal CO2 (ETCO2), mixed expired CO2 (PECO2), HR, and SpO2.

As indicated, the data gathering aspect of the disclosure involves known techniques and analyses, and the calculations for formulating predictive assessments are readily available in the scientific literature. However, by means of aspects of the feature extraction mechanism, classification and quantification scheme, the present disclosure enables an observer to gain new and valuable insight into the present condition and condition trends in patients. Thus, in accordance with a preferred method, a cardiopulmonary exercise gas exchange analysis is made for each test data set (see, e.g., FIG. 3). The performance of such a test is well understood by individuals skilled in the art, and no further explanation of this is believed necessary.

In such an example, reusable hardware, similar to that depicted in FIG. 1, can be used. Such hardware can include the analyzer, pulse oximeter module, a computer, and an optional printer. Disposable hardware that is used can include standard EKG electrodes and a Disposable Patient Interface including patient mouthpiece from Shape Medical Systems, Inc. of Saint Paul, Minnesota, and an analyzer bulkhead connector with three sample lines. Multiple EKG lead configurations are possible.

Next, at operation 1404, multiple metrics are analyzed based upon the captured patient data. As noted, this multi-variable approach provides an indication of the health and improvement or degradation of the patient's heart, lungs, and pulmonary vasculature. It can provide a more global picture of the patient's health over time and can be used, for instance, to determine how a therapy or drug regimen may be impacting the patient's health.

In this example, the following six metrics are analyzed:

MVI score-multivariable index, which integrates key gas exchange variables obtained during a submaximal exercise Test. This produces a severity score that ranges from normal (<1) to very severe (>=4), as described in U.S. Pat. No. 8,630,811; If the MVI decreases (Test 2−Test 1), add 1 to the EM2 score. If the score increases (Test 2−Test 1), add −1 to the EM² score. For no change add 0.

DR2—Disease Risk and Referral optimization, which provides a differential diagnosis based upon MVI and MPIph, as described in U.S. Pat. No. 10,010,264; If the DR2 decreases (Test 2−Test 1), add 1 to the EM² score. If the DR2 score increases (Test 2−Test 1), add −1 to the EM2 score. If no change, add zero.

MPIph—A multiparametric index of pulmonary hypertension, which provides a classification index for pulmonary hypertension, as described in U.S. Pat. Nos. 8,775,093 and 10,010,264; if the MPIph score decreases (Test 2−Test 1), add 1 to the EM² score. If the MPIph score increases (Test 2−Test 1), add −1 to the score. For no change, add zero.

VE slope—Minute Ventilation slope; if the VE slope decreases (Test 2−Test 1), add 1 to the EM² score. If it increases add −1 to the EM² score. For no change, add zero.

OUES—Oxygen Uptake Efficiency Slope, as provided in U.S. Pat. No. 8,630,811; if the QUES increases (Test 2−Test 1), add 1 to the EM² score. If it decreases add −1 to the EM² score. For no change, add zero.

GXcap—a correlation of pulmonary capacitance, as provided in U.S. Pat. No. 11,497,439; if the GXcap increases (Test 2−Test 1), add 1 to the EM² score. If it decreases add −1 to the EM² score. For no change, add zero.

In other examples, different metrics can be used. The example provided herein is simply one embodiment.

These metrics are then used at operation 1406 of the method 1400 to produce a single patient score, called EM², based upon the example metrics. As stated earlier, bundling of multiple metrics has been shown to enhance prognostic power. This EM² score can then be used as a guide to whether patient treatment, such as selected drugs, result in the patient's functional stability, improvement or worsening, as provided further below.

More specifically, the patient score is a number that is designed to give a healthcare professional one or more of: 1) a simple, objective indicator of the patient's current test status for each EM2 component variable (a score of +1 or −1) and 2) effectiveness of a patient's treatment (a cumulative score from −6 to +6). A positive cumulative score ≥2 shows positive results of treatment and could encourage the health professional to continue treatment unchanged. A negative cumulative score≤−2 shows the patient's condition is worsening and could be used by the health professional as reason to modify treatment.

For instance, referring now to FIGS. 15 and 16, example Tables 1500 and 1600 are shown listing the metrics used to calculate the patient score. In this example, each metric is provided on the Table 1500, and ranges are used to determine a value for each metric. Each value can range from −1 to +1. These values are then summed to arrive at a patient score ranging from −6 to +6.

FIG. 15 provides a detailed example of how a single test EM2 score can be used to derive a cumulative EM2 score (in the example, cumulative EM2=−6 if all EM2 variables show the patient worsening or +6 if all EM² variables are improving.

FIG. 16 shows the EM2 analysis performed for an actual patient test/retest (EM2=+4).

Each number in the Tables 1500 and 1600 is designed to give a healthcare professional a simple, objective indicator of the effectiveness of a patient's treatment. A positive number ≥2 shows positive results of treatment and could encourage the health professional to continue treatment unchanged. A negative score≤−2 shows the patient's condition is worsening and could be used by the health professional as reason to modify treatment.

Calculation of the EM² Score can be based upon the six components:

MVI score—multivariable index, which integrates 6 key gas exchange variables obtained during submaximal exercise into a severity score that ranges from normal (<1) to very severe (>=4), as described in U.S. Pat. No. 8,630,811; If the MVI decreases (Test 2−Test 1), add 1 to the EM² score. If the score increases (Test 2−Test 1), add −1 to the EM² score. For no change add 0

DR2-Disease Risk and Referral optimization, which provides a differential diagnosis based upon MVI, MPIph and other disease descriptive metrics, as described in U.S. Pat. No. 10,010,264; If the primary DR2 silo score decreases (Test 2−Test 1), add 1 to the EM² score. If the DR2 silo score increases (Test 2−Test 1), add −1 to the EM² score. If no change, add zero.

MPIph—A multiparametric index of pulmonary hypertension, which provides a classification index, based upon the ETCO2 profile/pattern throughout the test for pulmonary hypertension, as described in U.S. Pat. Nos. 8,775,093 and 10,010,264; if the MPIph score decreases (Test 2−Test 1), add 1 to the EM² score. If the MPIph score increases (Test 2−Test 1), add −1 to the score. For no change, add zero.

VE slope—Minute Ventilation efficiency slope; if the VE slope decreases (Test 2−Test 1), add 1 to the EM² score. If it increases add −1 to the EM² score. For no change, add zero.

QUES—Oxygen Uptake Efficiency Slope, as provided in U.S. Pat. No. 8,630,811; if the QUES increases (Test 2−Test 1), add 1 to the EM² score. If it decreases add −1 to the EM2 score. For no change, add zero.

GXcap—a strong correlation of right heart catheterization hemodynamically determined pulmonary capacitance, as provided in U.S. Pat. No. 11,497,439; if the GXcap increases (Test 2−Test 1), add 1 to the EM² score. If it decreases add −1 to the EM² score. For no change, add zero.

Finally, the values are summed to arrive at the single patient value (e.g., +4).

Referring again to FIG. 14, at operation 1408 of the method 1400, a patient assessment is provided based upon the metrics and patient score. In some examples, AI is used to provide the patient assessment.

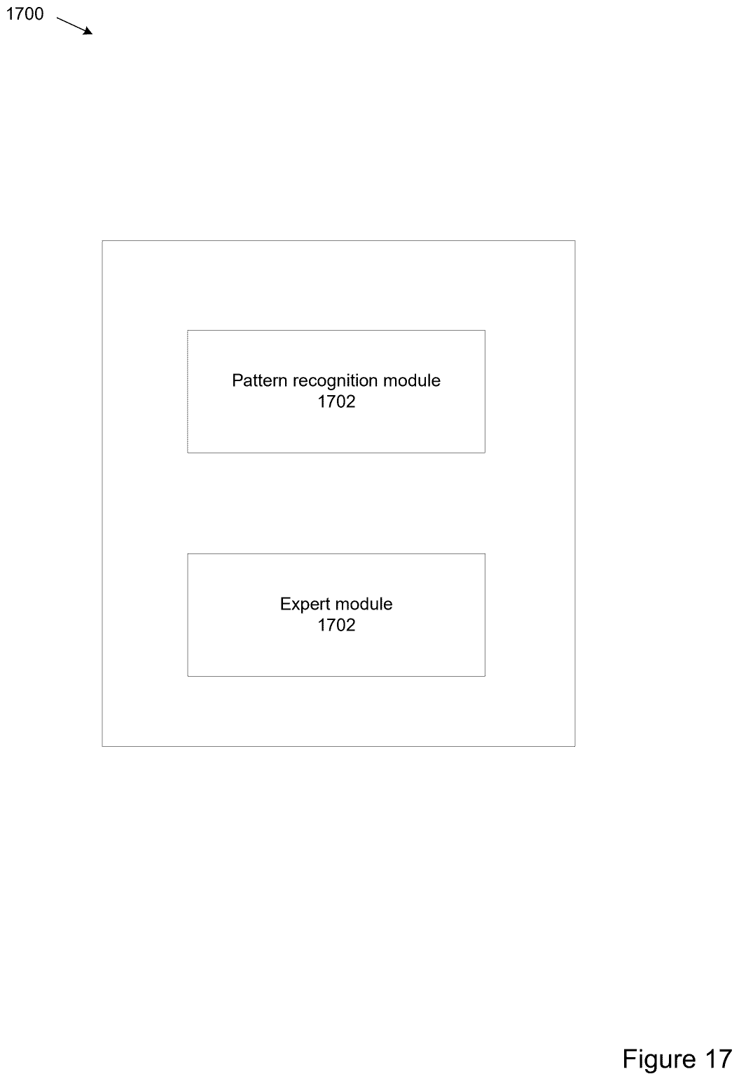

For example, referring now to FIG. 17, in one example, a system 1700 is programmed to provide a patient assessment based upon the metrics and patient score of the method 1500. More specifically, the system 1700 is programmed to provide the patient assessment based upon one or more of: a) a cardiopulmonary exercise gas exchange analyzer that gathers the observations to be classified or described; b) a feature extraction mechanism that computes numeric information from the observations; and c) a classification or description scheme that does the actual job of classifying or describing observations based on the extracted features. The system 1700 can thereby provide improvements in dyspnea diagnosis and monitoring through pattern recognition, which is the act of receiving raw data from the CPET metabolic analyzer and taking an action based on the category of the data.

In this embodiment, the system 1700 includes a pattern recognition module 1702 and an expert module 1704.

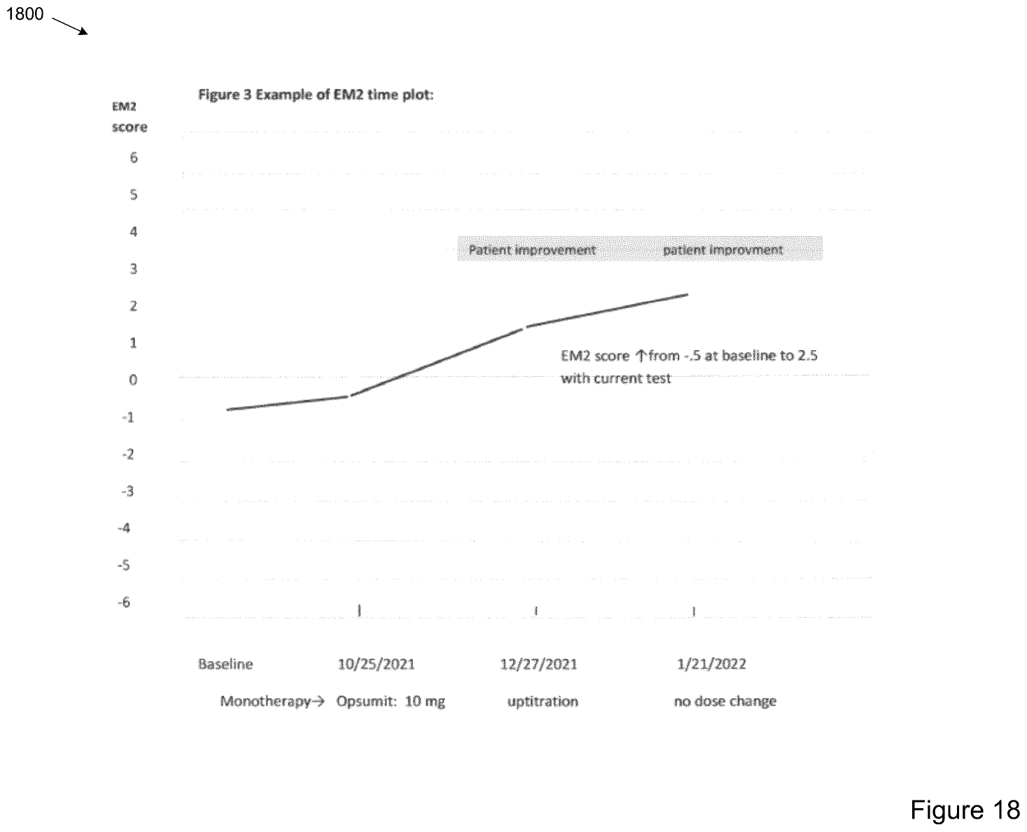

The example pattern recognition module 1702 is programmed to classify data patterns/signatures based on statistical information extracted from the patterns or signatures. The pattern recognition module 1702 can be programmed to: a) receive physiological measurements from the CPET metabolic analyzer that gathers the observations to be classified or described; b) compute numeric information from the observations; c) classify observations based on the extracted features; and d) provide an output to a user interface (see, e.g., FIG. 18).

The example expert module 1704 of the system 1700 utilizes the patterns recognized by the pattern recognition module 1702 to emulate the decision-making ability of a human expert in the interpretation of CPET data. The expert module 1704 is programmed to solve complex problems by reasoning through bodies of knowledge, represented mainly as “if-then” rules rather than through conventional procedural code. The example expert module 1704 can be divided into two subsystems: the inference engine and the knowledge base. The knowledge base represents facts and rules. The inference engine applies the rules to the known facts to deduce new facts (diagnosis and efficacy of treatment).

Referring now to FIG. 18, an example user interface 1800 is depicted. In this instance, the user interface 1800 can be generated by the system 1700.

The user interface 1800 provides a plot showing a series of patient scores for a patient over time. The user interface 1800 can display a trend based upon those scores, which allows a physician to readily see whether the patient is improving or degrading over time or stable.

For instance, in the example shown in FIG. 18, the user interface 1800 plots three patient scores, showing improvement from a negative value at the first score to a positive value at the third, most recent score. The slope of the line connecting these scores illustrates the patient's functional improvement or worsening over time.

The system 1700 can be programmed to provide certain statement triggers based upon the patient score and trending over time, along with other information about the patient. These triggers, in turn, are used to provide a guide for patient therapy management over time or even discern whether the quality of the test is adequate for validity of test data.

For instance, in one example, assume a 65 year old female patient with a history of ischemic heart disease (with previous placement of two stents), a pacemaker and COPD. The patient successfully performed a timed step test with adequate effort and quality. The example test performance metrics regarding test quality for the patient are the following:
Peak HR bpm: percentage age-predicted maximum (APM), if peak attained HR>70% APM supportive of good patient effort;
Peak RER: if peak attained RER >0.95, supportive of good patient effort;
% HR Reserve; if >50%, supports of good patient effort;
Borg Scale achieved: (if >4 supports good patient effort; and
Absence of HR and/or SpO2 dropout.

The system 1700 can use these patient metrics to generate a score and guidance to the physician as follows:

In one example, a trigger statement is based upon the aspects defined above and a minimum of two criteria being met with no unedited dropout for good sub-max test quality): if % APM HR achieved=75%; % HR reserve utilized=53%; peak attained respiratory exchange ratio (RER)=0.92 and Borg scale peak=4, then statement is: “With good patient effort, a timed step test was successfully performed with adequate quality.” In another example, if only one of the 5 criteria above were met with unedited HR or SpO2 dropout, then the statement is: “The patient performed a sub-optimal step test of inadequate quality.”

In other examples, the system 1700 can use other specific metrics found when calculating the patient DR2 or EM2 scores as triggers to provide guidance for patient care. The most abnormal metrics in these scores can be used as focused target metrics for therapy guidance for patient functional improvement.

If, for example, the primary DR2 silo score is cardiac, and the VE efficiency slope and delta exercise ETCO2 are both beyond their “cut-off” points, these two metrics can be targeted to watch for improvement with patient therapy management. Therefore, the triggered statement in the Impressions report could be an action item by the care provider, as such:” The ventilation efficiency slope and delta ETCO2, both represented in the EM² score, are target variables for improvement in the subsequent serial patient tests. A follow-up test within the next 4 to 6 months should be scheduled for therapy tracking and positive changes in these metrics.”

In yet other embodiments, the system 1700 can use specific clinical impressions as triggered statements to provide guidance for patient care.

In further embodiments, the system 1700 can use additional triggers. Another statement could be triggered from the rate response of a cardiac patient being inadequate based upon the patient having an implanted heart pacemaker that is possibly under-programmed or their sinus rhythm being inadequate due to beta blocker therapy. The triggered statement could be, as such: “The chronotropic response index slope was <0.7, indicating chronotropic incompetence during exercise. The patient's pacemaker could possibly be reprogrammed for restoring adequate rate response during exercise to improve cardiac output, O2 uptake and improve exercise tolerance.”

Another example could follow an EM2 plot that shows progressive decreases in the EM2 score in two follow-up tests that would generate a triggered Impressions report statement, as follows: “The EM2 score decreases in the two most recent tests, indicating patient worsening and the need for possible adjustment of patient therapy, either with additional medication combinations or drug dose up-titration.”

What is claimed is:

1. A computer-implemented method for diagnosing cause of exertional dyspnea, the method comprising:
receiving cardiopulmonary exercise test data corresponding to a submaximal cardiopulmonary exercise test performed by patients;
gathering, by a computing device, observations to be classified based upon cardiopulmonary exercise test data;
extracting, by the computing device, numerical information from the observations;
classifying, by the computing device, the numerical information; and
generating, by the computing device, an interpretation of the numerical information.

2. The computer-implemented method of claim 1, further comprising performing the gathering, the extracting, or the classifying uses artificial intelligence.

3. The computer-implemented method of claim 1, wherein the cardiopulmonary exercise test data includes two or more tests of: oxygen consumption; rate of oxygen consumption; carbon dioxide production; carbon dioxide exhalation; end tidal partial pressure of carbon dioxide exhalation; partial pressure of mixed expired carbon dioxide exhalation; partial pressure of mean expired carbon dioxide; heartrate; and oxygen saturation.

4. The computer-implemented method of claim 1, further comprising classifying the numerical information based upon one or more of: a multivariable index for disease risk, a disease referral optimization, such as a multiparametric index score for pulmonary hypertension, cardiac or obstructive/restrictive lung disease or mix thereof and a degree of patient de-conditioning due to a disease or level of fitness.

5. The computer-implemented method of claim 1, further comprising generating a visual representation of a functional performance of a patient.

6. The computer-implemented method of claim 5, wherein the visual representation is a graph illustrating a trend for the patient.

7. The computer-implemented method of claim 1, further comprising generating a response to therapy score for a patient based upon the numerical information.

8. The computer-implemented method of claim 7, wherein the response to the therapy score is generated by combining a multivariable index score, a disease risk and referral optimization score, and other select Gx based functional variables.

9. The computer-implemented method of claim 7, further comprising using the therapy score to generate a guide for treating the patient.

10. The computer-implemented method of claim 9, wherein the guide indicates a positive or a negative reaction to medication or other treatment by the patient.

11. A computing device programmed for diagnosing cause of exertional dyspnea, the computing device comprising:
at least one processor; and
memory encoding instructions which, when executed by the at least one processor, cause the computing device to:
receive cardiopulmonary exercise test data corresponding to a submaximal cardiopulmonary exercise test performed by patients;
gather observations to be classified based upon the cardiopulmonary exercise test data;
extract numerical information from the observations;
classify, by the computing device, the numerical information; and
generate an interpretation of the numerical information.

12. The computing device of claim 11, comprising further instructions which, executed by the at least one processor, cause the computing device to perform the gathering, the extracting, or the classifying uses artificial intelligence.

13. The computing device of claim 11, wherein the cardiopulmonary exercise test data includes two or more tests of: oxygen consumption; rate of oxygen consumption; carbon dioxide production; carbon dioxide exhalation; end tidal partial pressure of carbon dioxide exhalation; partial pressure of mixed expired carbon dioxide exhalation; partial pressure of mean expired carbon dioxide; heartrate; and oxygen saturation.

14. The computing device of claim 11, comprising further instructions which, executed by the at least one processor, cause the computing device to classify the numerical information based upon one or more of: a multivariable index for disease risk, a disease referral optimization, such as a multiparametric index score for pulmonary hypertension, cardiac or obstructive/restrictive lung disease or mix thereof and a degree of patient de-conditioning due to a disease or level of fitness.

15. The computing device of claim 11, comprising further instructions which, executed by the at least one processor, cause the computing device to generate a visual representation of a functional performance of a patient.

16. The computing device of claim 15, wherein the visual representation is a graph illustrating a trend for the patient.

17. The computing device of claim 11, comprising further instructions which, executed by the at least one processor, cause the computing device to generate a response to a therapy score for a patient based upon the numerical information.

18. The computing device of claim 17, wherein the response to the therapy score is generated by combining a multivariable index score, a disease risk and referral optimization score, and other select Gx based functional variables.

19. The computing device of claim 17, comprising further instructions which, executed by the at least one processor, cause the computing device to use the therapy score to generate a guide for treating the patient.

20. The computing device of claim 19, wherein the guide indicates a positive or a negative reaction to medication or other treatment by the patient.
